(19) United States
(12) Patent Application Publication
(10) Pub. No.: US 2004/0267358 A1
(43) Pub. Date: Dec. 30, 2004

(54) Implant for treating an insufficiency of a heart valve

(76) Inventor: Oyvind Reitan, Lund (SE)

Correspondence Address: HARNESS, DICKEY & PIERCE, P.L.C., P.O. BOX 828, BLOOMFIELD HILLS, MI, 48303, US

(21) Appl. No.: 10/865,260

(22) Filed: Jun. 10, 2004

Related U.S. Application Data
(63) Continuation of application No. PCT/EP02/13439, filed on Nov. 28, 2002.

(30) Foreign Application Priority Data
Dec. 11, 2001 (DE) 101 61 543.4

(51) Int. Cl. A61F 2/24

(52) U.S. Cl. 623/2.37; 623/902

(57) ABSTRACT

An implant for treating insufficiency of a heart valve, in particular of the mitral valve, has an elongate body which can be converted from a first, substantially elongated and substantially flexible state to a second state with a reduced bending radius. The implant also has a flexible tensioning element extending along the body for the purpose of converting the body from the first state to the second state. The body is made up of a plurality of elements which are arranged in series to form a chain and which, in the first state, are movable relative to one another. In the second state of the body, the tensioning element tensions the elements with respect to one another so as form an arc, said elements being substantially immovable relative to one another in the second state and forming, on an internal radius face of the arc, a substantially continuous planar support surface.

### CROSS REFERENCE TO OTHER APPLICATIONS

The present application is a continuation of pending international patent application PCT/EP02/13439 filed on Nov. 28, 2002 which designates the United States and published in German and which claims priority of German patent application 101 61 543.4 filed on Dec. 11, 2001.

### BACKGROUND OF THE INVENTION

The invention relates to an implant for treating an insufficiency of a heart valve, in particular of the mitral valve.

Cardiovascular diseases account for a large share of the mortality rate in the western world.

Heart failure can have causes other than myocardial infarction; for example, it can be a result of high blood pressure over a long period of time or a result of metabolic or hereditary diseases.

The heart diseases which can lead to heart failure also include insufficiency of the heart valves, in particular of the mitral valve.

The heart consists of two halves, namely the right half and the left half. Both halves of the heart each consist of an atrium and a ventricle. The ventricles are the actual heart pumps, while the atria constitute a filling mechanism for the ventricles. The right ventricle pumps the blood through the lesser circulation, i.e. through the lungs for gas exchange, while the left ventricle pumps the blood through the whole body.

A widening (dilation) of the heart is often a sign of acute or chronic failure of the left ventricle. Dilation which occurs gradually is considered as a compensation phenomenon designed to obtain more force and represents a particular property of the heart. This widening is not limited to the left ventricle only; it also often leads to widening of the ring between the left atrium and the left ventricle. The mitral valve is secured on this ring. The mitral valve is a heart valve situated between the left atrium and the left ventricle. This heart valve consists of two cusps, an anterior cusp and a posterior cusp, which prevent blood from flowing back from the ventricle into the atrium.

The cusps are designed for a certain ring diameter. When the ring enlarges, the size of the cusps is no longer sufficient to ensure a tight closure of the flap valve, and leakage occurs between the two cusps, with the blood escaping backwards on each pumping movement, which can lead to heart failure. In most cases, it is mainly the posterior cusp which is responsible for the leakage, since the anterior cusp has a better anchoring through the soft skeleton of the heart.

In surgical treatment of a dilation of the mitral valve ring, it is possible to shorten the ring so that the cusps again adapt to the original size and, consequently, the mitral valve closes sealingly again. In order to reduce the size of the ring, a mitral valve replacement, for example a metal ring, is often fitted surgically into the mitral valve ring. However, such an intervention can only be performed by open-heart surgery, which imposes a considerable physical burden on the patient. In many cases, however, patients whose condition has already been impaired by heart failure cannot be operated on, since the mortality rate in these operations is too high. For these patients, the only remaining option then is drug treatment, but this is often not enough.

There is therefore a need for new treatment forms which impose less of a physical burden on the patients and in which the mortality rate is considerably reduced. In other words, there is a great need for treating insufficiency of a heart valve by a minimally invasive procedure.

WO 01/00111 A1 discloses an implant for treating insufficiency of the mitral valve by a minimally invasive procedure. The latter exploits the fact that the principal vein of the heart, the coronary sinus, runs in the posterior sulcus between the left atrium and the left ventricle. This course of the coronary sinus is followed exactly by the posterior curvature of the mitral valve ring in immediate proximity to the vein. The attachment of the posterior cusp of the mitral valve is located along the coronary sinus and covers approximately half the ring circumference. By means of an implant introduced into the coronary sinus by an intravascular route, that is to say using a minimally invasive procedure, it is thus possible to shorten the circumference of the posterior arc of the ring or to stiffen and stabilize the ring.

WO 01/00111 A1 describes various embodiments of implants intended for this purpose.

A first type of the known implant has a body designed as a vascular stent which is implanted in the coronary sinus. The vascular stent has a diameter corresponding to the diameter of the coronary sinus. The vascular stent is made of a wire latticework. The fact that the material used for the wires has a temperature-dependent shape memory means that, after insertion of the vascular stent into the coronary sinus, said stent assumes a state in which it has a reduced bending radius. However, a disadvantage of designing the implant as a vascular stent is that a stent implanted in the coronary sinus can lead to clotting and thrombosis and can thus reduce the blood circulation in the heart muscle. Moreover, the wires of the vascular stent, which bear on the vessel wall of the coronary sinus, can lead to rupturing of the wall. The fact that the reduction in radius is based solely on the shape memory of the wires used means that in some circumstances the stabilizing action of the known implant is also insufficient to stabilize the ring of the mitral valve. In addition, the conversion of the body from the substantially elongated state to the second state with smaller bending radius, which is based only on the material properties of the wires, cannot be adequately controlled.

A second type of the known implant consists simply of a wire which is introduced into the coronary sinus. In this wire, the change from the first, substantially elongated state to the second state with reduced bending radius takes place likewise on the basis of a shape memory of the wire material used, resulting once again in the abovementioned disadvantages. Since the conversion of the wire from the first state to the second state is also temperature-dependent, it is possible that the wire will undesirably curve too early during its introduction through the vessel system, as a result of which it is impossible to advance the wire farther.

In a third type of the known implant, the body consists of three short stent portions which are spaced far apart from one another and which each have a latticework and are interconnected by tensioning elements in the form of pull wires. After introduction of the three stent portions into the coronary sinus, the tensioning elements are tensioned by pulling them, by which means the distance between the three stent portions is slightly reduced and the radius of the mitral valve ring is made smaller. However, the disadvantage here is that the tensioning elements, which lie exposed between the stent portions, straighten during tensioning and can cut into the vessel wall of the coronary sinus.

### SUMMARY OF THE INVENTION

It is one object of the invention to provide an implant that is easy to implant.

It is another object of the invention to provide an implant in which the change from the first state to the second state can be easily controlled.

It is another object of the invention to provide an implant which ensures sufficient stabilizing of the ring of the heart valve.

It is another object of the invention to provide an implant which prevents damaging of the wall of the vessel into which it is implanted.

According to the invention, an implant for treating an insufficiency of the heart valve is provided, comprising an elongate body convertable from a first, substantially elongated and substantially flexible state to a second state in which the body has a reduced bending radius. The body is made up of a plurality of elements arranged in series to form a chain and, in the first state, being movable relative to one another. A flexible tensioning element extends along the body for the purpose of converting the body from the first state to the second state, wherein the tensioning element tensions the elements the respect to one another so as to form an arc. The elements are substantially immovable relative to one another in the second state and form, on an internal radius face of the arc, a substantially continuous planar support surface.

According to the invention, this object is achieved, in respect of the implant mentioned at the outset, by the fact that the body is made up of a plurality of elements which are arranged in series to form a chain and which, in the first state, are movable relative to one another, and that, in the second state of the body, the tensioning element tensions the elements with respect to one another so as to form an arc, said elements being substantially immovable relative to one another in the second state and forming, on an internal radius face of the arc, a substantially continuous planar support surface.

Accordingly, the implant according to the invention has a body which is made up of a plurality of elements which are arranged in series to form a chain and which, when the tensioning element is not tensioned, are movable relative to one another, so that the implant, in the first state of the body, can be introduced into the vessel or coronary sinus by an intravascular approach. As soon as the implant is positioned in situ in the vessel, the tensioning element is tensioned, as a result of which the elements are tensioned so as to form an arc and thus bear with a substantially continuous planar support surface on the wall of the vessel facing toward the ring of the heart valve and effectively support the ring of the heart valve. The implant does not cut into or press into the vessel wall, because it is possible to define a curvature of the arc which follows the natural curvature of the vessel, without giving rise to straightened portions. In the second state of the body, the mutually tensioned elements form an arc which, depending on the configuration of the elements and of the tensioning element, can be stiff or rigid, or the arc can also have a certain elasticity. In this sense, the feature according to which the elements in the second state are “substantially immovable” relative to one another is to be understood as meaning that, if the ring is rigid or stiff, the elements are completely immovable or, if the arc still has a certain flexibility, they still have a slight mobility relative to one another or are even flexible. The implant according to the invention can, in the first state of the body, be easily introduced by an intravascular route, for example by means of a catheter, and, when in situ in the vessel, can then be tensioned in a controlled manner by means of the tensioning element, for example by means of the catheter used for the introduction, as a result of which, upon suitable actuation of the tensioning element, the element-stension and form the arc.

In a preferred embodiment, the tensioning element is arranged on a side of the elements which is directed away from the support surface.

The advantage of this is that it ensures that, in contrast to the implants known from the prior art, the tensioning element does not straighten and cut into the vessel wall during tensioning. The side of the elements directed away from the support surface is not only to be understood as meaning that the tensioning element is arranged, for example, on the reverse side of the elements, but that, in the case of a hollow design of the elements, the tensioning element can also be integrated into the elements and extend through the interior of the elements.

In a further preferred embodiment, respectively adjacent elements are connected to one another in an articulated manner.

This measure has the advantage that the individual elements are already connected to one another in the first state of the body, as a result of which the conversion to the second state, through tensioning of the tensioning element, can take place with even better control. In the first state of the body, the articulated connection of respectively adjacent elements has the advantage that, during the intravascular introduction into the vessel, the body made up of said elements has the necessary flexibility to be able to adapt to the course of the vessel.

In a preferred embodiment, provision is made that the articulated connection between adjacent elements is formed by flexible connecting portions between the elements.

This measure has the advantage that the articulated connection can be realized in a structurally simple manner. These connecting portions can be formed in one piece with the individual elements and, for example, upon production of the individual elements, they can be formed from a single piece of material by leaving material bridges in place between the individual elements.

In a preferred alternative embodiment, the articulated connection between adjacent elements is formed by axle hinges.

Although this measure represents a more complicated embodiment of the connection of the elements, it nevertheless has the advantage that the articulated connection between the individual elements can be made very stable as a whole. In this embodiment, individually produced elements can be connected to one another via the axle hinges to form the body of the implant.

In a further preferred embodiment, the tensioning element is secured on the distal element and is connected to the proximal element via a tensioning mechanism which permits continuous tensioning of the tensioning element.

By means of this measure, the conversion of the body of the implant according to the invention from the first state to the second state can be even better controlled; in particular, the bending radius of the arc in the second state of the body can, by means of the continuous tensioning of the tensioning element, be adapted very precisely to the particular site of application and individually from patient to patient.

In a further preferred embodiment, the tensioning mechanism has a screw thread which is arranged on the proximal element and with which a proximal portion of the tensioning element can be engaged by screwing.

With a suitably fine configuration of the screw thread, the embodiment of the tensioning mechanism with a screw thread arranged on the proximal element permits a continuous tensioning of the tensioning element, with a possibility of very fine adaptation of the second state of the body of the implant. The tensioning mechanism is preferably actuated using the catheter with which the implant was brought to the target site by the intravascular route, in which case such a catheter is then equipped with a suitable shaft which can be maneuvered by the operating surgeon from outside the body in order to tension the tensioning element and convert the body to the second state.

In a further preferred embodiment, the elements have, on their side facing toward the heart valve, an eyelet for passing through the tensioning element.

This measure has the advantage that the tensioning element is guided on the elements. In this embodiment, it is also possible to dispense with an interconnection of the elements, as is preferably provided for in one of the preceding embodiments, since in this case the elements on the tensioning element can be secured against twisting in rotation about their longitudinal direction preferably by the eyelet.

In a further preferred embodiment, an anchoring hook is arranged on at least one element, on the support surface side.

The anchoring hook serves to catch in the tissue of the heart valve ring, by which means the implant according to the invention is advantageously secured against slipping or displacement in the implanted state.

In a further preferred embodiment, the anchoring hook can be extended out beyond the support surface.

This measure has the advantage that the at least one anchoring hook does not impede the intravascular insertion, because it can be arranged in a recessed position in which it does not constitute a hindrance. It is only at the target site that the anchoring hook is then extended outward past the support surface of the body, affording the further advantage that the anchoring hook catches securely in the tissue of the ring when it is extended.

In a further preferred embodiment, the anchoring hook is pivotable, and the tensioning element is connected to the anchoring hook in order to pivot it outward.

This measure then has the particular advantage that, upon tensioning of the tensioning element, the at least one anchoring hook is extended automatically past the support surface of the element on which it is provided. The extension movement of the at least one anchoring hook takes place at the start of tensioning of the tensioning element, as a result of which the implant is first of all anchored at the target site, and, by further tensioning of the tensioning element, the body is then converted to the second state in which said body is deformed to provide the arc shape. The added advantage of this measure is that only one actuating mechanism, namely the tensioning element, is necessary for the extension movement of the anchoring hook and for converting the body from the first state to the second state.

In a further preferred embodiment, the anchoring hook is arranged at least on the distal element, and the tensioning element is secured on the distal element via the anchoring hook.

In accordance with the aforementioned embodiment, this measure has the advantage that the tensioning element is in this way connected both to the distal element, for converting the body from the first state to the second state, and also to the at least one anchoring hook for extension of the latter.

However, it is also preferable if an anchoring hook is arranged in each case on several elements.

Not only does this achieve even better fixation of the implant in the coronary sinus, the body can then also be formed from the elements in such a way that, in the second state, it still has a certain flexibility or elasticity, for example by means of the individual elements of the body still having a certain relative mobility with respect to one another or being themselves flexible.

In a further preferred embodiment, the elements have a curvature at least on their outside forming the support surface.

This measure has the advantage that the elements are from the outset equipped with a suitable preliminary curvature, so that the support surface, in the second state of the implant body formed by the elements, has a curvature adapted to the anatomical curvature of the ring that is to be stabilized. This embodiment is advantageous in particular when the individual elements, and thus also preferably the body, are flexurally stiff or even rigid in the second state.

However, it is likewise preferable if such a curvature is given to the individual elements only at the time of implantation, for example under the effect of the body temperature, by using a suitable material for the elements, for example nitinol.

In a further preferred embodiment, end faces of the elements are designed in such a way that the end faces of adjacent elements lie flush on one another in the second state of the body.

The advantage of this is that, with the body in the implanted state, the end faces of the elements do not constitute an obstacle to the flow of blood running through the vessel and thus do not cause the blood to form eddies. Moreover, the support surface, which is formed by the outside of the elements facing toward the heart valve in the second state of the body, is continuously smooth, as a result of which the vessel wall of the coronary sinus is still better protected by the implant.

In a further preferred embodiment, the elements are substantially flexurally stiff.

The advantage of this is that, when tensioned to constitute the arc, the elements can form an overall flexurally stiff or even rigid partial ring which ensures particularly good stabilizing of the dilated natural ring and, thus, of the heart valve that is to be treated.

In a further preferred embodiment, the elements have, in cross section, a wall extending all the way round their circumference.

This measure has the advantage that, as is provided for in another preferred embodiment, it allows the tensioning element to be arranged in the interior of the elements which are then designed as hollow bodies.

It will be appreciated that the cross-sectional shape is not restricted to a circular configuration, and that other cross-sectional shapes may be preferred instead, for example cross sections of crescent shape or semicircular cross sections, which have the further advantage that the implant in the vessel takes up a smaller cross section of the vessel and ensures an adequate flow of blood through the coronary sinus.

In a further preferred embodiment, the wall of the elements is at least partially interrupted.

By means of this measure, the surface of the implant can be still further reduced, as a result of which the danger of a thrombus formation or the danger of coagulation can be further minimized.

It is likewise preferable if the elements have, in cross section, a wall extending only part of the way round their circumference.

In this embodiment of the individual elements with an open cross section, the tensioning element of course cannot be completely surrounded by the elements; however this measure has the advantage that the cross section of the vessel taken up by the implant can be kept very small, so that the passage of blood through the coronary sinus is impaired to the least possible extent.

In a further preferred embodiment, a surface of the elements is anti-adhesive and biocompatible with respect to biological tissue or blood.

This measure has the advantage that the implant according to the invention is biocompatible as such, and the danger of thrombus formation can be avoided. The individual elements can for example be coated with a suitable material, or the elements can be made from a suitable material, or the body of the implant can be sheathed with a suitable flexible, thin tubing which covers the individual elements.

Further advantages and features will become clear from the following description and from the attached drawing.

It will be appreciated that the aforementioned features and those still to be explained below can be used not only in the respectively cited combination, but also in other combinations, or in isolation, without departing from the scope of the present invention.

### BRIEF DESCRIPTION OF THE DRAWINGS

Illustrative embodiments of the invention are set out in the drawing and are described below in greater detail with reference to said drawing, in which:

FIG. 1 shows an implant for treating insufficiency of a heart valve, in a first state;

FIG. 2 shows the implant from FIG. 1 in a second state;

FIG. 3 shows a cross section through the implant from FIG. 1 long the line III-III in FIG. 1;

FIG. 4 shows a partial representation of the implant from FIG. 1, in a view from behind;

FIG. 5 shows a distal end of the implant from FIGS. 1 and 2 on an enlarged scale, and partially in longitudinal section;

FIG. 6 shows the implant from FIG. 1 together with an auxiliary instrument, which is represented very diagrammatically,

FIG. 7 shows a proximal end of the implant from FIG. 1 on an enlarged scale in longitudinal section;

FIG. 8 shows the implant from FIG. 1 in the implanted state, with a heart valve represented very diagrammatically;

FIG. 9 and FIG. 10 show further illustrative embodiments of geometries of elements for an implant in cross section.

### DETAILED DESCRIPTION OF PREFERRED EMBODIMENTS

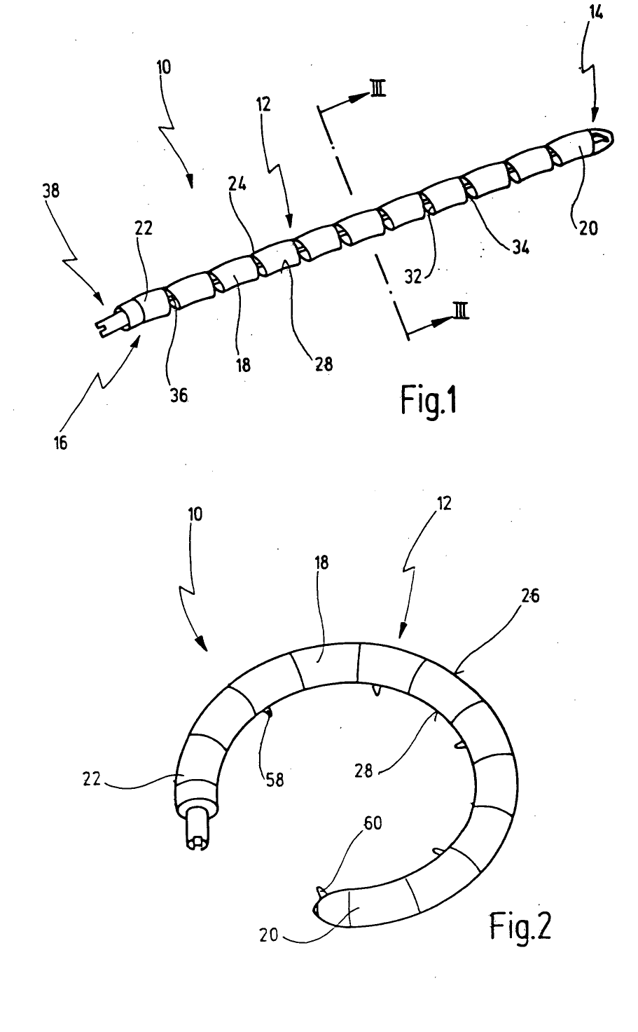

In FIGS. 1, 2, 6 and 8, an implant for treating insufficiency of a heart valve, in the present case for treating insufficiency of the mitral valve, is designated by the general reference numeral 10. The implant 10 is used for stabilizing the natural ring, present on the mitral valve, in cases of pathological dilation or slackening of the ring.

The implant 10 generally has a body 12 comprising a distal end 14 and a proximal end 16.

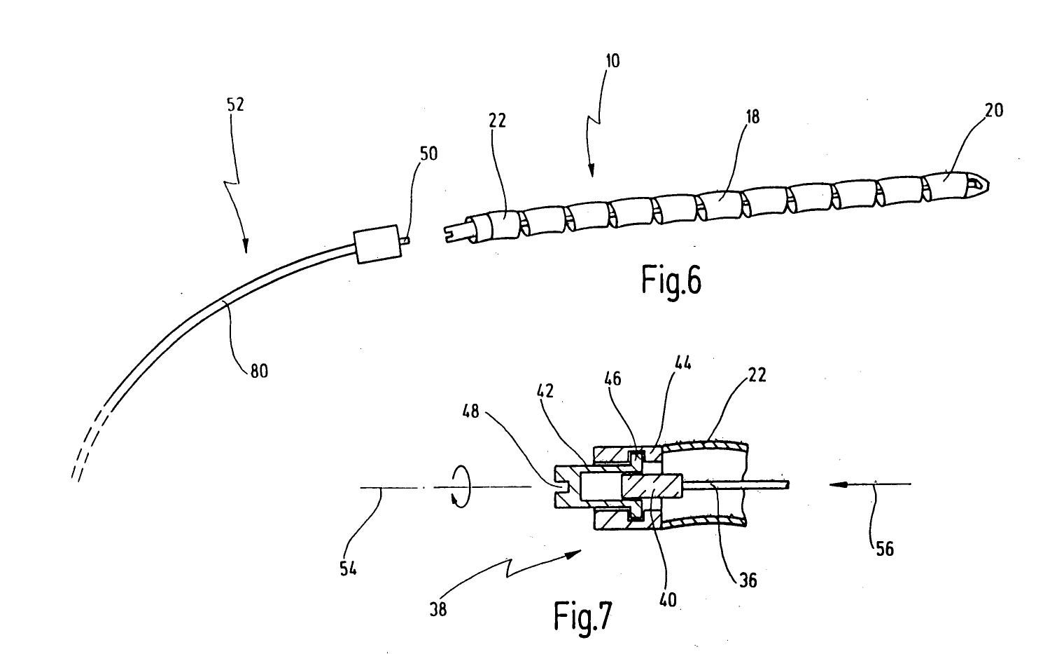

In FIGS. 1 and 6, the body 12 is shown in a first state in which it is substantially elongated and substantially flexible, so that in this first state it can be advanced along arteries or veins toward its implantation site by an intravascular route by means of a catheter and in so doing can easily adapt even to the curved course of these vessels.

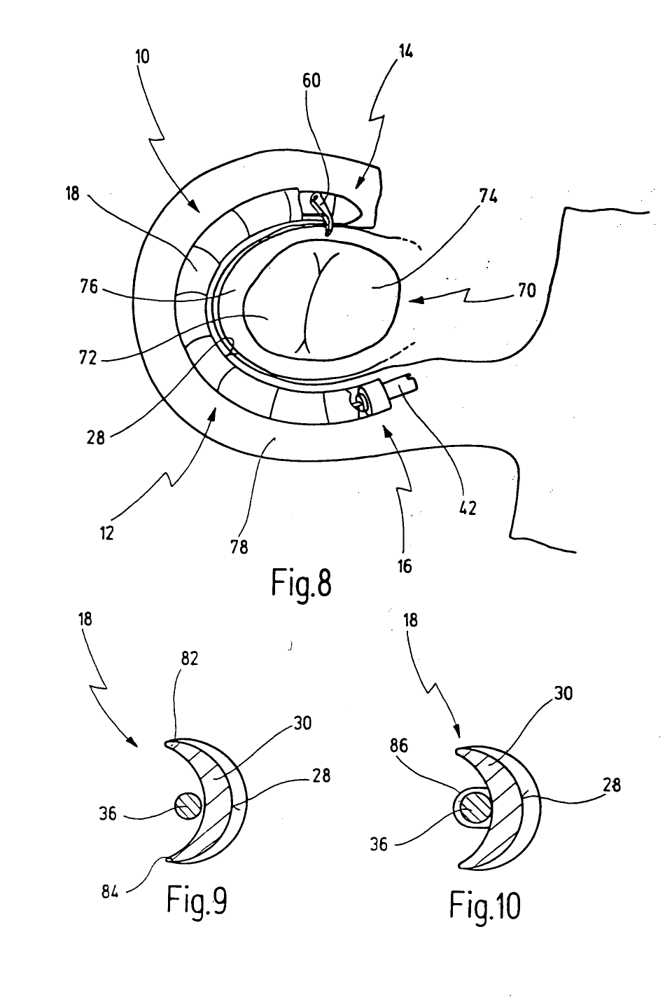

In FIGS. 2 and 8, the body 12 is shown in a second state in which it assumes a curved shape with a reduced bending radius. In this state, the implant 10 is able to exert its stabilizing function for the natural ring of the heart valve that is to be treated.

The body 12 is made up of a plurality of elements 18 which are arranged in series to form a chain. In the illustrative embodiment shown, the body 12 is made up of a total of eleven such elements 18, a distal element 20 forming the distal end 14 of the body 12, and a proximal element 22 forming the proximal end 16 of the body 12.

In the first state of the body 12 represented in FIGS. 1 and 6, the elements 18 are movable relative to one another. In the illustrative embodiment shown, the relative mobility is provided by the fact that respectively adjacent elements 18 are connected to one another in an articulated manner. By this means, the elements 18 are movable in a direction transverse to the longitudinal direction of the body 12, but substantially immovable relative to one another in the longitudinal direction of the body 12.

The articulated connection of respectively adjacent elements 18 is formed by flexible connecting portions 24 between the elements 18. The connecting portions 24 are arranged eccentrically with respect to the longitudinal center axis of the body 12.

However, instead of being connected by flexible connecting portions, the individual elements 18 of the body 12 can also be connected to one another in an articulated manner via axle hinges.

The articulated connection of the individual elements 18 is effected on a side of the body 12 which, in the second state of the body 12 according to FIG. 2, forms an external radius face 26, while the elements 18 are not connected to one another on an internal radius face 28 lying opposite the external radius face 26.

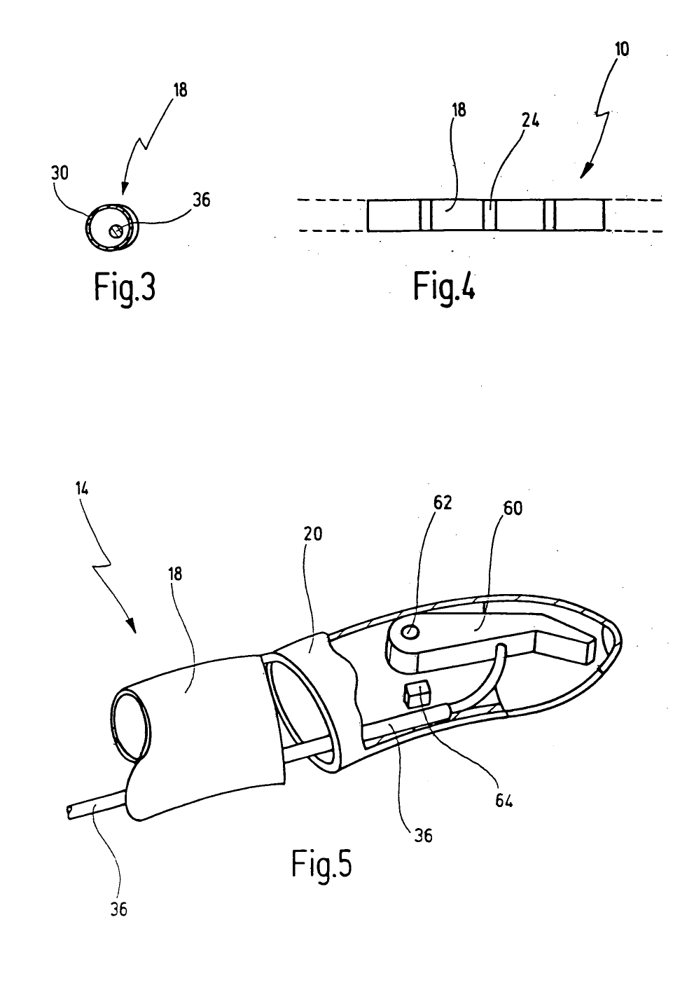

The elements 18 have, in cross section (cf. FIG. 3), a wall 30 extending all the way round their circumference, i.e. the individual elements 18 are designed in the form of small tubes. Each element 18 thus represents a hollow body with a wall 30 extending all the way round its circumference. At end faces 32 and 34, each element 18 is open in the longitudinal direction.

The end faces 32 and 34 of the elements 18 are designed in such a way that the end faces 32 and 34 of adjacent elements 18 lie flush on one another in the second state of the body ¹², as is shown in FIGS. 2 and 8. In the embodiment of the individual elements 18 as tubes, the body 12 thus assumes the form of a substantially continuously closed partial ring along its longitudinal direction.

To produce the body 12, the latter can be produced, for example, from a tube shaped as a partial ring according to FIG. 2, by partially cutting into the tube wall from the internal radius face 28 transversely with respect to the longitudinal center axis of the tube, the flexible connecting portions 24 being left in place in the cutting procedure. When the tube thus formed is stretched out, the configuration of the body 12 according to FIG. 1 is then obtained.

In the second state of the body 12 as shown in FIGS. 2 and 8, the elements 18 form, on the internal radius face 28, a substantially continuous planar support surface which, in the implanted state of the implant 10, is directed toward the heart valve to be treated.

In order to convert the body 12 from the first state shown in FIGS. 1 and 6 to the second state shown in FIGS. 2 and 8, a flexible tensioning element 36 is provided which extends along the body 12. The tensioning element 36 is, for example, a thin wire, which for example can be made of steel, nitinol or other suitable materials.

The tensioning element 36 extends from the proximal end 16 to the distal end 14 of the body 12. The tensioning element 36 is arranged on that side of the elements 18 directed away from the internal radius face 28 forming the support surface, and it runs through the inside of the elements 18.

The tensioning element 36 is secured on the distal element 20 and is connected to the proximal element 22 via a tensioning mechanism 38, which will be described in more detail below with reference to FIG. 7.

An element 40 with an external thread is arranged at the proximal end of the tensioning element 36 and is in threaded engagement with a screw sleeve 42 having an internal thread. The screw shank 42 for its part is able to turn relative to the proximal element 22 but is axially immovable, for which purpose a sleeve 44 is fixedly connected to the proximal element 22, into which sleeve 44 a radial projection 46 of the screw sleeve 42 engages.

Cut into the outermost proximal end of the screw sleeve 42 there is a slit 48 into which a corresponding auxiliary instrument 52 can be fitted which, at its distal end, has a corresponding projection 50, as is shown in FIG. 6.

By turning the screw sleeve 42 about a longitudinal axis 54, the tensioning element 36 in threaded engagement with the screw sleeve 42 is pulled proximally in the direction of an arrow 56. Since the tensioning element 36 is secured on the distal element 20, the elements 18 are accordingly tensioned with respect to one another to form an arc according to FIG. 2 and according to FIG. 8. In the state in which they have been tensioned to form the arc, the elements 18 are then held on one another by the tensioning element 36 and are substantially immovable relative to one another.

The tensioning mechanism 38 permits a continuous tensioning of the tensioning element 36.

While the body 12 in FIGS. 2 and 8 is shown in the maximally tensioned state, in which the end faces 32 and 34 of adjacent elements 18 lie completely on one another, it is likewise possible to tension the body 12 only partially, by which means the body 12 can form an arc which has a greater bending radius than in FIGS. 2 and 8.

Moreover, an anchoring hook is arranged on at least one of the elements 18, on the internal radius face 28 of the body 12, with five such anchoring hooks 58 being provided according to FIG. 2. The anchoring hooks 58 can be extended outward past the internal radius face 28, forming the support side, of the body 12, the outward extension movement of the anchoring hooks 58 taking place during tensioning of the tensioning element 36, as is described in more detail with reference to FIG. 5 which shows the example of an anchoring hook 60 arranged on the distal element 20.

The tensioning element 36 is secured on the distal element 20 via the anchoring hook 60. The anchoring hook 60 is mounted on the distal element 20 in such a way as to be able to pivot about a pivot axis 62. Upon tensioning of the tensioning element 36 by turning the screw sleeve 42, the anchoring hook 60 is first of all extended outward or pivoted out from the inwardly pivoted position in the distal element 20, as shown in FIG. 5. A limit stop 64 limits the maximum pivoting of the anchoring hook 60. As soon as the anchoring hook 60 bears on the limit stop 64, further tensioning of the tensioning element 36 then ensures that the body 12 is converted to the second state, in which it assumes the arc shape according to FIGS. 2 and 8.

As can be seen from FIG. 1, the elements 18 already have a curvature on their outside forming the support surface or internal radius face 28, which curvature corresponds to the bending radius of the body 12 in the state shown in FIG. 2. If the elements 18 are in addition of a substantially flexurally stiff or even rigid design, the body 12 in the second state shown in FIG. 2 forms a substantially flexurally stiff or even rigid arc or partial ring.

The surface of the elements 18 is anti-adhesive and biocompatible with respect to biological tissue or blood, and this can be achieved by a suitable coating or by a flexible tubular sheath. However, the elements 18 can also be made altogether of a biocompatible material. The elements 18 can also be made of metal and be provided with a suitable coating or a tubular sheath.

Moreover, the wall 30 of the elements 18 can be provided with continuous perforations in order to additionally reduce the surface in this way.

FIG. 8 is a diagrammatic representation showing the implant 10 in the implanted state.

Reference numeral 70 here designates the mitral valve, which has a posterior cusp 72 and an anterior cusp 74. The illustration in FIG. 8 corresponds to a plan view of the mitral valve 70. The mitral valve is arranged on the heart between the left atrium and the left ventricle. The mitral valve 70 is secured in place by a natural ring 76. The ring 76 may be pathologically widened (dilated) or slackened, with the result that the mitral valve 70 no longer closes sealingly during the pumping movements of the heart.

The implant 10 now serves to re-establish the closing action of the mitral valve 70 by virtue of the fact that the implant 10 sufficiently supports and strengthens the natural ring 76 or reduces its radius.

For this purpose, the implant 10 is implanted into the coronary sinus 78 which surrounds the ring 76 and which represents the principal vein of the heart muscle and is immediately adjacent to the ring 76.

In the first state of the body according to FIGS. 1 and 6, the implant 10 is introduced into the coronary sinus using a catheter system, with the femoral vein, for example, serving as an access. The femoral vein is also used in heart examinations with a Swan-Ganz catheter. By means of a guide catheter, the implant 10 can then be positioned in the coronary sinus 78 by radioscopy. The implant 10 is secured on an auxiliary catheter which has an inner channel and is provided with a rotatable shaft, as is shown for example in FIG. 6, for the auxiliary instrument 52, by reference numeral 80. The projection 50 of the auxiliary instrument 52 according to FIG. 6 engages in the slit 48 of the screw sleeve 42. By turning the shaft 80, the screw sleeve 42 is then set in rotation, by which means the tensioning element 36 is pulled in the proximal direction according to the direction of the arrow 56 in FIG. 6. The at least one anchoring hook 60 and, if appropriate, the other anchoring hooks 58 also are first extended outward and catch in the tissue of the ring 76. By further turning of the shaft 80, the tensioning element 36 is then pulled farther in the proximal direction, as a result of which the body 12 converts from the first state to the second state according to FIG. 2 and

FIG. 8, in which the elements 18 form the arc or partial ring which, with the internal radius face 28 forming the support surface, bears on the vessel wall of the coronary sinus 78 adjacent to the ring 76 and in this way supports the ring 76.

The arc formed by the elements 18 covers a circle angle of preferably more than 180°, by which means the implant 10 engages as far as possible round the ring 76 and is additionally stabilized in its position.

When the elements 18 are tensioned completely with respect to one another, the auxiliary instrument 52 detaches from the screw sleeve 42 and can then be withdrawn from the body with the aid of the auxiliary catheter. Instead of a tensioning mechanism with screw thread, another tensioning mechanism can also be used, for example a bayonet-type mechanism.

The cross-sectional dimension of the implant 10 should be as small as possible to ensure that the largest possible lumen of the coronary sinus 78 remains available for the passage of blood.

FIGS. 9 and 10 show examples of other cross-sectional shapes of the elements 18. The cross-sectional shape shown in FIGS. 9 and 10 is approximately crescent-shaped, in which case the tensioning element 36 is not surrounded by the elements 18 but instead lies free. In the illustrative embodiment shown in FIGS. 9 and 10, the wall 30 of the elements 18 thus extends in cross section round only part of the circumference.

In the case of an articulated connection to one another, the elements 18 can then have flexible connecting portions at their cross-section ends 82 and 84.

According to FIG. 10, it is also possible, with this “open” construction of the elements 18, for the tensioning element 36 to be threaded through eyelets 86 on the individual elements 18, in which case it is also possible to dispense with a connection of the elements 18 to one another.

What is claimed is:

1. An implant for treating an insufficiency of a heart valve, comprising:
an elongate body convertable from a first, substantially elongated and substantially flexible state to a second state in which said body has a reduced bending radius, wherein said body is made up of a plurality of elements arranged in series to form a chain and, in said first state, being movable relative to one another,
a flexible tensioning element extending along said body for the purpose of converting said body from said first state to said second state, wherein said tensioning element tensions said elements with respect to one another so as to form an arc, said elements being substantially immovable relative to one another in said second state and forming, on an internal radius face of said arc, a substantially continuous planar support surface.

2. The implant of claim 1, wherein said tensioning element is arranged on a side of said elements which is directed away from said support surface.

3. The implant of claim 1, wherein respectively adjacent ones of said elements are connected to one another in an articulated manner.

4. The implant of claim 3, wherein said articulated connection between said adjacent elements is formed by flexible connecting portions between said elements.

5. The implant of claim 3, wherein said articulated connection between said adjacent elements is formed by axle hinges.

6. The implant of claim 1, wherein said tensioning element is secured on a distal element of said elements and is connected to a proximal element of said elements via a tensioning mechanism which permits continuous tensioning of said tensioning element.

7. The implant of claim 6, wherein said tensioning mechanism has a screw thread which is arranged on said proximal element and with which a proximal portion of said tensioning element can be engaged by screwing.

8. The implant of claim 1, wherein said elements have an eyelet for passing through said tensioning element.

9. The implant of claim 1, wherein an anchoring hook is arranged on at least one of said elements, on said support surface side.

10. The implant of claim 9, wherein said anchoring hook can be extended out beyond said support surface.

11. The implant of claim 10, wherein said anchoring hook is pivotable, and said tensioning element is connected to said anchoring hook in order to pivot said anchoring hook outward.

12. The implant of claim 11, wherein said anchoring hook is arranged at least on a distal element of said elements, and wherein said tensioning element is secured on said distal element via said anchoring hook.

13. The implant of claim 1, wherein a plurality of said elements have an anchoring hook arranged thereon.

14. The implant of claim 1, wherein said elements have a curvature at least on their outside forming said support surface.

15. The implant of claim 1, wherein end faces of said elements are designed in such a way that said end faces of adjacent ones of said elements lie flush on one another in said second state of said body.

16. The implant of claim 1, wherein said elements are substantially flexibly stiff.

17. The implant of claim 1, wherein said elements have, in cross-section, a wall extending all the way round their circumference.

18. The implant of claim 17, wherein said elements are open at end faces thereof, and said tensioning element runs through an inside of said elements.

19. The implant of claim 17, wherein said wall of said elements is at least partially interrupted.

20. The implant of claim 1, wherein said elements have, in cross-section, a wall extending only part of the way round their circumference.

21. The implant of claim 1, wherein a surface of said elements is anti-adhesive and biocompatible with respect to biological tissue and blood.
